(19) United States
(12) Patent Application Publication
(10) Pub. No.: US 2021/0114958 A1
(43) Pub. Date: Apr. 22, 2021

(54) APPARATUS AND METHOD FOR PRODUCING METHANOL

(71) Applicant: AVOCET IP LTD, Northumberland (GB)

(72) Inventors: James Robert JENNINGS, North Yorkshire (GB); Glyn David SHORT, Hockessin, DE (US)

(21) Appl. No.: 16/464,400

(22) PCT Filed: Nov. 27, 2017
(86) PCT No.: PCT/EP2017/025346

§ 371 Date: May 28, 2019

(30) Foreign Application Priority Data
Nov. 27, 2016 (GB) 1620034.7

(51) Int. Cl. C07C 29/151; C12M 1/107; B01D 53/047; C01B 3/38

(52) U.S. Cl. CPC C07C 29/1518; C12M 21/04; C07C 31/04; C01B 3/382; B01D 53/047

(57) ABSTRACT

An apparatus is provided for producing methanol from organic material, wherein the apparatus includes: (i) an anaerobic digestion arrangement for receiving the organic material and for anaerobically-digesting the organic material in oxygen-depleted conditions to generate an anaerobic digestion gas (AD gas) comprising at least methane, and carbon dioxide; (ii) a pressure swing absorption (PSA) arrangement for the removal of excess carbon dioxide; (iii) a chemical reaction arrangement for reacting the methane gas with water vapour and carbon dioxide in a stoichiometric condition of the reaction, CO2+3CH4+2H2O=4CH3OH, between methane steam reforming and methane dry reforming to generate a synthesis gas, and converting the synthesis gas to methanol; and (iv) an recovery arrangement for recovering unreacted methane and feeding the recovered unreacted methane into the exit stream from the anaerobic digestion arrangement.

### TECHNICAL FIELD

The present disclosure relates to methods of producing methanol, for example to methods of producing methanol from organic waste material, for example agricultural organic waste. Moreover, the present disclosure also relates to apparatus that are operable to implement aforementioned methods. Furthermore, the present disclosure relates to computer program products comprising a non-transitory computer-readable storage medium having computer-readable instructions stored thereon, the computer-readable instructions being executable by a computerized device comprising processing hardware for executing aforementioned methods.

### BACKGROUND

In overview, methanol (CH₃OH) is a liquid fuel at room temperature (i.e. at circa 20° C.) that is storable in steel tanks, being relatively non-corrosive in nature. Methanol is not highly toxic, although a mere 30 cm³ to 100 cm³ quantity of methanol can be lethal if ingested. It is less dangerous than gasoline if inhaled, and far less toxic than two popular household cleaning fluids, namely trichloroethylene and carbon tetrachloride.

It is known that methanol is corrosive to certain materials in a vehicle's fuel system, for example aluminium components. Contemporary metal floats and synthetic cements employed in vehicle manufacture resist a solvent action exhibited by methanol. Iron and steel are quite immune to corrosion from methanol, as are also brass and bronze alloys.

Methanol is potentially a highly valuable energy carrier, because it can be combusted in contemporary combustion engines to provide mechanical power, and can also be oxidized in fuel cells to provide electrical power. Moreover, the oxidation of methanol results in the generation of carbon dioxide and water vapour that are regarded as benign to the environment.

Although methanol is a major, product of the petrochemicals industries with an annual tonnage well in excess of 100 million tonnes per annum, it has not found general significant use in transport, heating buildings and aviation because its volume-to-energy density is less than that of petrol, diesel oil and kerosene. Thus, for many industrial processes, methanol has not been used as extensively as possible.

Bulk production of methanol in a conventional methanol plant typically involves a steam reforming stage for the preparation of synthesis gas. During conversion, a portion of the methane typically escapes from the converter unreacted, ultimately reducing the yield of methanol per production cycle.

Conventional solutions to such a problem of involve installing a purge stream, for example for feeding the methane to a fuel stream that is burned to sustain the steam reformer or for being recycled into the methane gas feed. However, such conventional solutions do not increase the production yield as these solutions tackle the initial loss of methane by either recycling or burning the methane gas, both resulting in lower yield per production cycle.

With growing environmental concerns, despite considerable “unseen” pollution from nuclear power plants and similar industrial sites occurring, there is contemporary concern to recycle waste products from industry and farming to reduce their environmental impact, as the World struggles to try to achieve a greater degree of long-term sustainability in its commercial activities. Agricultural waste is potentially an environmental issue that has caused concern more recently. In particular, it is desirable to convert agricultural waste that is otherwise a cost overhead into a valuable commercial by-product.

### SUMMARY

The present disclosure seeks to provide an improved method of generating methanol, for example from biological waste, for example agricultural waste.

Moreover, the present disclosure seeks to provide an improved apparatus for implementing aforementioned improved methods.

According to a first aspect, there is provided an apparatus for producing methanol in a continuous manner from organic material, characterized in that the apparatus includes:

(i) an anaerobic digestion arrangement for receiving the organic material and for anaerobically-digesting the organic material in oxygen-depleted conditions to generate an anaerobic digestion gas (AD gas) comprising at least methane, and carbon dioxide;
(ii) a pressure swing absorption (PSA) arrangement for the removal of excess carbon dioxide;
(iii) a chemical reaction arrangement for simultaneously reacting the methane gas with water vapour and carbon dioxide in a stoichiometric condition of reaction

CO₂+3CH₄+2H₂O=4CH₃OH

between methane steam reforming and methane dry reforming within at least one vessel to generate a synthesis gas, and converting the synthesis gas to methanol; and
(iv) a recovery arrangement for recovering unreacted methane and feeding the recovered unreacted methane into the exit stream from the anaerobic digestion arrangement. Optionally, the apparatus includes an arrangement for feeding hydrogen into the exit stream from the anaerobic digestion arrangement.

Optionally, that the arrangement for feeding hydrogen generates hydrogen by a photocatalytic process.

Optionally, the stoichiometric condition is maintained using a control arrangement, provided in operation with temperature sensing signals and gas component sensing signals indicative of operating conditions within the chemical reaction arrangement, for controlling rates of supply of the methane gas, water vapour and carbon dioxide into the chemical reaction arrangement.

Optionally, the chemical reaction arrangement is operable to employ a catalyst arrangement including, nickel, nickel alumina, nickel foil, copper and/or platinum catalysts.

Optionally, the chemical reaction arrangement is operable to provide the stoichiometric condition of reaction

CO₂+3CH₄+2H₂O=4CH₃OH:

(i) at a first stage for steam reforming at a pressure in a range of 10 Bar to 30 Bar, and at a temperature in a range of 750° C. to 950° C.; and
(ii) at a second stage of methanol synthesis at a pressure in a range of 50 Bar to 150 Bar, and at a temperature in a range of 200° C. to 250° C.

According to a second aspect, there is provided a method of using an apparatus for producing methanol in a continuous manner from organic material, characterized in that the method includes:

(i) receiving the organic material in an anaerobic digestion arrangement, and anaerobically-digesting the organic material in oxygen-depleted conditions to generate a gas comprising methane and carbon dioxide;
(ii) removing excess carbon dioxide in a pressure swing absorption (PSA) arrangement;
(iii) simultaneously reacting the gas with water vapour and carbon dioxide in a stoichiometric condition of reaction

CO₂+3CH₄+2H₂O=4CH₃OH

between methane steam reforming and methane dry reforming within at least one vessel_to generate a synthesis gas and converting the synthesis gas to methanol in a chemical reaction arrangement (30); and
(iv) recovering unreacted methane in a recovery arrangement and feeding the recovered unreacted methane into the exit stream from the anaerobic digestion arrangement (20).

Optionally, the method includes maintaining the stoichiometric condition using a control arrangement, provided in operation with temperature sensing signals and gas component sensing signals indicative of operating conditions within the chemical reaction arrangement, for controlling rates of supply of the methane gas, water vapour and carbon dioxide into the chemical reaction arrangement.

Optionally, the method includes using a renewable energy source for providing operating power to the chemical reaction arrangement.

Optionally, the method includes operating the chemical reaction arrangement to employ a catalyst arrangement including nickel-alumina, nickel foil, copper and/or platinum catalysts.

Optionally, the method includes operating the chemical reaction arrangement (30) to provide the stoichiometric condition of reaction

CO₂+3CH₄+2H₂O=4CH₃OH:

(i) at a first stage for steam reforming at a pressure in a range of 10 Bar to 30 Bar, and at a temperature in a range of 750° C. to 950° C.; and
(ii) at a second stage of methanol synthesis at a pressure in a range of 50 Bar to 150 Bar, and at a temperature in a range of 200° C. 5 to 250° C.

According to a third aspect, there is provided a computer program product comprising a non-transitory computer-readable storage medium having computer-readable instructions stored thereon, the computer-readable instructions being executable by a computerized device comprising processing hardware for executing a method of the first aspect.

The invention is of advantage in that operating substantially at the stoichiometric condition (Eq. 4) allows for highly efficient production of methanol, based on biogas supplied from an anaerobic digester supplied for organic material, for example organic agricultural waste. The invention is of further advantage of maximising the potential yield of methanol by preventing any stoichiometric imbalance during steam reforming. Moreover, embodiments of the present invention advantageous in terms of significantly reducing amount of bi-products formed during production of methanol despite of operating the chemical reaction arrangement at low temperature and using low cost and/or less active catalysts.

It will be appreciated that features of the invention are susceptible to being combined in various combinations without departing from the scope of the invention as defined by the appended claims.

### BRIEF DESCRIPTION OF THE DIAGRAMS

Embodiments of the present disclosure will now be described, by way of example only, with reference to the following diagrams wherein:

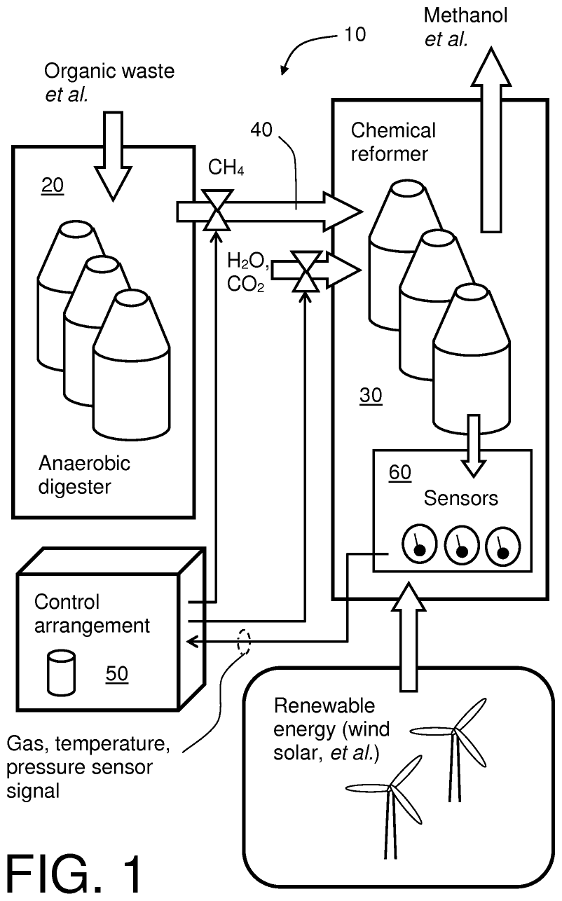

FIG. 1 is an illustration of an apparatus for producing methanol pursuant to the present disclosure; and

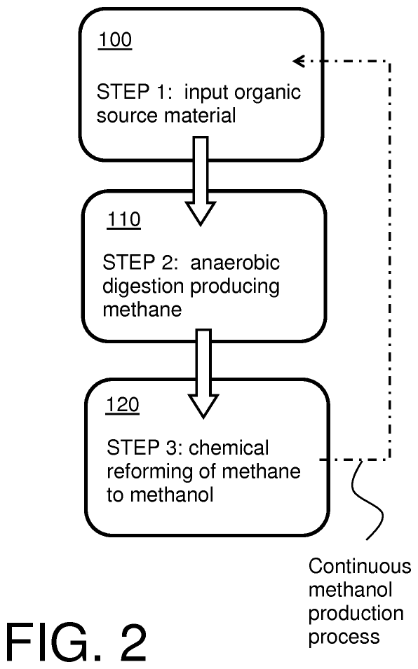

FIG. 2 is an illustration of steps of a method of producing methanol using the apparatus of FIG. 1.

In the accompanying diagrams, an underlined number is employed to represent an item over which the underlined number is positioned or an item to which the underlined number is adjacent. When a number is non-underlined and accompanied by an associated arrow, the non-underlined number is used to identify a general item at which the arrow is pointing.

### DETAILED DESCRIPTION OF EMBODIMENTS

According to a first aspect, there is provided an apparatus for producing methanol from organic material, characterized in that the apparatus includes:

(i) an anaerobic digestion arrangement for receiving the organic material and for anaerobically-digesting the organic material in oxygen-depleted conditions to generate an anaerobic digestion gas (AD gas) comprising at least methane, and carbon dioxide;
(ii) a pressure swing absorption (PSA) arrangement for the removal of excess carbon dioxide;
(iii) a chemical reaction arrangement for reacting the methane gas with water vapour and carbon dioxide in a stoichiometric condition (Eq. 4) between methane steam reforming and methane dry reforming to generate a synthesis gas, and converting the synthesis gas to methanol; and
(iv) an recovery arrangement for recovering unreacted methane and feeding the recovered unreacted methane into the exit stream from the anaerobic digestion arrangement.

Optionally, the apparatus for producing methanol from organic material includes an arrangement for feeding hydrogen into the system.

Optionally, the apparatus for producing methanol from organic material includes an arrangement for generating hydrogen.

Optionally, the arrangement for generating hydrogen generates hydrogen by a photocatalytic process

The invention is of advantage in that operating substantially at the stoichiometric condition (Eq. 4) allows for highly efficient production of methanol, based on biogas supplied from an anaerobic digester supplied for organic material, for example organic agricultural waste. The invention is of further advantage of maximising the potential yield of methanol by preventing any stoichiometric imbalance during steam reforming.

Optionally, the apparatus for producing methanol from organic material includes a unit for recovering unreacted methane and feeding the recovered unreacted methane into the exit stream from the anaerobic digestion unit.

Optionally, the apparatus for producing methanol from organic material includes a unit for recovering unreacted methane and feeding the recovered unreacted methane into the exit stream from the anaerobic digestion unit.

Optionally, the apparatus for producing methanol from organic material includes a unit for feeding hydrogen into the system.

Optionally, the apparatus for producing methanol from organic material includes a unit for generating hydrogen.

Optionally, the unit for generating hydrogen generates hydrogen by a photocatalytic process

Optionally, the unit for generating hydrogen is a fuel cell.

Optionally, in the apparatus, the stoichiometric condition is maintained using a control arrangement, provided in operation with temperature sensing signals and gas component sensing signals indicative of operating conditions within the chemical reaction arrangement, for controlling rates of supply of the methane gas, water vapour and carbon dioxide into the chemical reaction arrangement.

Optionally, the apparatus includes a renewable energy source for providing operating power to the chemical reaction arrangement.

Optionally, in the apparatus, the chemical reaction arrangement is operable to employ a catalyst arrangement including nickel-alumina, nickel foil, copper-zinc-alumina and/or platinum catalysts.

Optionally, in the apparatus, the chemical reaction arrangement is operable to provide the stoichiometric condition (Eq. 4):

(i) at a first stage for steam reforming at a pressure in a range of 10 Bar to 30 Bar, and at a temperature in a range of 750° C. to 950° C.; and
(ii) at a second stage of methanol synthesis at a pressure in a range of 50 Bar to 150 Bar, and at a temperature in a range of 200° C. to 250° C.

Optionally, the apparatus is operable to produce methanol in a continuous manner.

According to a second aspect, there is provided a method of using an apparatus for producing methanol from organic material, characterized in that the method includes:

(i) receiving the organic material in an anaerobic digestion arrangement, and anaerobically-digesting the organic material in oxygen-depleted conditions to generate methane gas;
(ii) removing excess carbon dioxide in a pressure swing absorption (PSA) arrangement;
(iii) reacting the gas with water vapour and carbon dioxide in a stoichiometric condition (Eq. 4) between methane steam reforming to generate a synthesis gas and converting the synthesis gas to methanol in a chemical reaction arrangement; and
(iv) recovering unreacted methane in a recovery arrangement and feeding the recovered unreacted methane into the exit stream from the anaerobic digestion arrangement.

Optionally, the method includes maintaining the stoichiometric condition using a control arrangement, provided in operation with temperature sensing signals and gas component sensing signals indicative of operating conditions within the chemical reaction arrangement, for controlling rates of supply of the methane gas, water vapour and carbon dioxide into the chemical reaction arrangement.

Optionally, the method includes using a renewable energy source for providing operating power to the chemical reaction arrangement.

Optionally, the method includes operating the chemical reaction arrangement to employ a catalyst arrangement including nickel-alumina, nickel foil, copper and/or platinum catalysts.

Optionally, the method includes operating the chemical reaction arrangement to provide the stoichiometric condition (Eq. 4):

(i) at a first stage for steam reforming at a pressure in a range of 10 Bar to 30 Bar, and at a temperature in a range of 750° C. to 950° C.; and
(ii) at a second stage of methanol synthesis at a pressure in a range of 50 Bar to 150 Bar, and at a temperature in a range of 200° C. to 250° C.

Optionally, the method includes operating the apparatus to produce methanol in a continuous manner.

According to a third aspect, there is provided a computer program product comprising a non-transitory computer-readable storage medium having computer-readable instructions stored thereon, the computer-readable instructions being executable by a computerized device comprising processing hardware for executing a method of the first aspect.

In overview, the present disclosure is concerned with a method of processing organic waste in an anaerobic digestion arrangement to provide methane gas, and then to reform the methane gas to generate corresponding methanol. Energy for implementing the method beneficially is provided from renewable energy resources, for example solar cells, heliostats, wind turbines, hydroelectric turbines (for example, micro-turbines inserted into small streams and rivers).

To prevent creating excess hydrogen and thus the need for hydrogen recovery during methanol production, carbon dioxide recovered by a pressure swing absorption (PSA) arrangement, which allows methane to go through the unit unreacted.

Typical anaerobic digestion gas (AD gas) contain excess of carbon dioxide, for example an AD gas composition with 60% carbon dioxide. The excess of carbon dioxide may be compensated by an injection of hydrogen, which in turn, may be obtained via an external source, for example a photocatalytic production unit or, for example, a fuel cell.

The above method/steps applies mutatis mutandis for situations in which the removal of carbon dioxide (by any method) creates an excess of hydrogen gas. In the present disclosure, any excess carbon dioxide gas may be recovered by the PSA unit. Alternatively, the excess of carbon dioxide may be corrected by an injection of hydrogen.

In the present disclosure, even if in a first reacting cycle, or few reacting cycles, a portion of the methane remains unreacted, the unreacted methane is recovered by the methane recovery arrangement, and fed into the exit stream of the anaerobic digestion arrangement or directly into the chemical reaction arrangement for converting the synthesis gas to methanol.

In particular, wherein carbon dioxide and the unreacted methane are continuously recovered, respectively by the PSA arrangement, and the methane recovery arrangement, and, the carbon dioxide and the unreacted methane are fed into the exit stream of the anaerobic digestion arrangement prior to the PSA system, the build-up of carbon dioxide would be removed while simultaneously cleaning the AD gas. The methane content would be recovered as would any carbon monoxide and hydrogen and this would go into the steam reformer feed gas.

Among other advantages, any, even if slight, imbalance in the carbon dioxide removal stage, which could result in an excess of either hydrogen or carbon oxides in the methanol ‘production cycle’ (also referred as ‘synthesis loop’), would be continuously controlled without any loss of the valuable methane, and therefore, resulting in a more efficient production cycle. The present invention maximises the potential yield of methanol with little or any additional cost to either the capital installation or running costs.

The method includes a concurrent combination of:

(i) steam reforming of methane to generate methanol and an excess of hydrogen; and
(ii) dry reforming of methane with carbon dioxide to generate methanol and an excess of carbon monoxide,

wherein a combination of (i) and (ii) in a correct stochiometric proportion is operable to produce a gas mixture that is optimal for purposes of methanol synthesis.

Chemical reactions associated with (i) and (ii) will next be described in greater detail.

In a first stage, organic waste (for example, livestock waste, animal slurry, cellulose plant-harvest waste, denatured fruit and vegetables and similar that are unsuitable for sale for human consumption or for animal feed) and/or organic crop material (for example, maize) is provided to an anaerobic digester arrangement wherein, in an oxygen-depleted environment, microorganisms are operable to convert the organic waste and/or organic crop material into methane and other reaction by-products.

In the anaerobic digester arrangement, there is employed a collection of processes by which microorganisms break down biodegradable material in the absence of oxygen. Such a process is contemporarily used for industrial or domestic purposes to manage waste, or to produce fuels. The processes are akin, in many respects, to fermentation that is used industrially to produce food and drink products. It will be appreciated that anaerobic digestion occurs naturally in some soils and in lake and oceanic basin sediments, where it is usually referred to as “anaerobic activity”. This is the source of marsh gas methane as discovered by a scientist Volta in year 1776.

In the aforementioned anaerobic digester arrangement, there occurs in operation a digestion process that begins with bacterial hydrolysis of input materials provided to the anaerobic digester arrangement, for example agricultural waste as aforementioned. Insoluble organic polymers, such as carbohydrates, are broken down to soluble derivatives (including sugars and amino acids) that become available for other bacteria that are present in the anaerobic digester arrangement. Thereafter, acidogenic bacteria then convert the sugars and amino acids into carbon dioxide gas, hydrogen gas, ammonia gas and organic acids. Moreover, these acidogenic bacteria convert these resulting organic acids into acetic acid, along with additional ammonia gas, hydrogen gas, and carbon dioxide gas. Finally, methanogens convert such gaseous products to methane and carbon dioxide. Thus, such methanogens, for example methanogenic archaea populations, play an indispensable role in anaerobic wastewater treatments that are feasible to achieve using the aforementioned anaerobic digester arrangement.

The anaerobic digestion arrangement is operable to function as a source of renewable energy, for example for producing biogas, consisting of a mixture of methane, carbon dioxide and traces of other trace gases. This biogas can be used directly as fuel, in combined heat and power gas engines or upgraded to natural gas-quality bio-methane. There is also generated from the anaerobic digestion arrangement a nutrient-rich digestate that can be used as a fertilizer.

In practice, the anaerobic digestion arrangement includes at least one closed vessel, for example fabricated from welded steel sheet, and is provided with a screw-feed arrangement for introducing, for example in a continuous manner, the aforementioned organic waste and/or organic crop material into the at least one closed vessel. Anaerobic digestion processes occurring within the at least one vessel result in an excess gaseous pressure to arise within the at least one vessel, wherein biogas can be selectively vented from the at least one vessel to provide biogas feedstock to a subsequent process. Beneficially, a screw-feed arrangement is used to remove digestate, for example in a continuous manner, from a lower region of the at least one vessel.

In embodiments of the present disclosure, the biogas feedstock is provided to a chemical reforming arrangement that will next be described in greater detail. The chemical reforming arrangement is beneficially implemented as a two-stage process involving:

(i) a first stage of steam reforming; and
(ii) a second stage of methanol synthesis.

The stages are optionally implemented in a single reaction vessel. Alternatively, the stages are optionally implemented in two or more reaction vessels. Beneficially, when two or more reaction vessels are employed, a first reaction vessel is operable to accommodate in operation steam reforming and a second reaction vessel is operable to accommodate in operation methanol synthesis.

A plurality of controllable gas feeds is provided to the at least one reaction vessel, for example two or more reaction vessels, including a gas feed for the aforementioned biogas from the anaerobic digestion arrangement. The at least one reaction vessel is provided with a gas sensing arrangement, for example implemented using one or more infrared radiation absorption gas analysers and/or electrochemical gas analysers, for measuring a stoichiometry of gases present in operation within the at least in one reaction vessel. Optionally, the at least one reaction vessel is provided with a catalyst arrangement, for example for the second stage, for example for both first and second stages, for example a metal mesh arrangement (for example fabricated from Nickel Alumina, Nickel foil, Platinum, Copper or similar), and a source of heat.

The source of heat is optionally supplied from renewable energy resources, for example spatially geographical local to the chemical reforming arrangement (for example, as would be appropriate for off-grid implementations of embodiments of the present disclosure when implemented in a rural environment, for example when operated in rural Latin America, rural India, rural Middle East, on isolated islands and such like).

For the first stage of steam reforming, there is utilized an internal pressure in the at least one vessel in a range of 5 Bar to 50 Bar, and more optionally in a range of 10 Bar to 30 Bar. Moreover, for the first stage of steam forming, the at least one reaction vessel is, for example, optionally operated having an internal operating temperature in a range of 300° C. to 1200° C., more optionally an internal operating temperature in a range of 750° C. to 950° C. When implementing the first stage of steam forming, there is beneficially provided an excess of hydrogen (H₂) for the steam reforming reaction.

For the second stage of methanol synthesis, there is utilized an internal pressure in the at least one vessel in a range of 30 Bar to 150 Bar, and more optionally in a range of 50 Bar to 100 Bar. Moreover, for the second stage of methanol synthesis, the at least one reaction vessel is, for example, optionally operated having an internal operating temperature in a range of 150° C. to 300° C., more optionally an internal operating temperature in a range of 200° C. to 250° C. Preferably, operating temperatures in excess of 260° C. are avoided, as they tend to result in a formation of metallic nanoparticles, for example copper nanoparticles, on catalyst surfaces that can be detrimental to throughput of synthesis of methanol during the second stage. The second stage, in operation results in an excess of carbon dioxide (CO₂) that is reacted with excess hydrogen (H₂) from the first stage.

A processor-based control arrangement is provided and is operable to monitor and control the stoichiometric composition of gases within the at least one reaction vessel (for example a single vessel, two vessels, and so forth, as aforementioned) the internal operating temperature of the at least one reaction vessel, the internal pressure of the at least one reaction vessel, gas mixing occurring within the at least one reaction vessel (for example flows of steam, biogas and carbon dioxide (for example a degree of turbulence in mixing), and optionally a temperature of a catalyst arrangement present within the at least one reaction vessel.

Chemical reactions occurring within the at least one reaction vessel are primarily concerned with converting biogas provided from the anaerobic digestion arrangement, namely principally methane, into methanol. Beneficially, the at least one reaction vessel is heated with energy supplied from renewable energy sources, for example wind turbine, solar panels and so forth.

In methane steam reforming processes, as employed for the first stage, there is generated an excess of hydrogen (H₂), relative to the amount of carbon oxides generated for methanol synthesis; such a methane steam reforming process is represented by Equation 1 (Eq. 1):

CH₄+H₂O═CO+3H₂=CH₃OH+H₂ Eq. 1

However, in methane dry reforming processes, as employed for the second stage, there is produced a gas that is deficient in hydrogen (H₂) for methanol synthesis, relative to the amount of residual carbon oxides; such a methane dry reforming process is represented by Equation 2 (Eq. 2):

2CH₄+2CO₂=4CO+4H₂=2CH₃OH+2CO Eq. 2

The aforementioned at least one reaction vessel of the chemical reforming arrangement employs a combination of operating conditions that lie between regimes represented by Equation 1 (Eq. 2) and Equation 2 (Eq. 2). A combination of the two regimes represented by Equation 1 (Eq. 1) and Equation 2 (Eq. 2) in a correct proportion is operable to produce a gas mixture that is just optimal for purposes of methanol synthesis.

Thus, in the chemical reforming arrangement, the following two reactions pertain simultaneously within the at least one vessel (Eqs. 3A, 3B), for example two or more vessels:

CO₂+3H₂=CH₃OH+H₂O Eq. 3A

3CH₄+3H₂O=3CH₃OH+3H₂ Eq. 3B

Thus, when the stoichiometry of gaseous reactants present in operation within the at least one reaction vessel is appropriately controlled, there is derived by addition that a chemical reaction as provided by Equation 4 (Eq. 4) is achieved:

CO₂+3CH₄+2H₂O=4CH₃OH Eq. 4

When stoichiometry is achieved, an amount of hydrogen (H2) and carbon dioxide generated (CO2) at the first and second stages is substantially matched, for example to within at least 10%, more optionally to within at least 5%, and yet more optionally to within at least 1%.

From the foregoing, it will be appreciated that if biogas generated by the anaerobic digestion arrangement is only slightly upgraded from its raw state of circa 60% methane and 40% carbon dioxide to exactly 75% methane and 25% methane, then steam reforming with an appropriate excess of steam is capable of producing an exactly stoichiometric synthesis gas required for efficient methanol manufacture. Appropriate reaction conditions are required, as described in the foregoing.

In an exemplary embodiment, the apparatus for producing methanol from organic material may include an anaerobic digestion arrangement for receiving the organic material and for anaerobically-digesting the organic material in oxygen-depleted conditions to generate a methane-containing AD gas; a chemical reaction arrangement for reacting the methane gas with water vapour and carbon dioxide in a stoichiometric condition (Eq. 4) between methane steam reforming and methane dry reforming to generate methanol synthesis gas; and a methanol synthesis arrangement for converting the methanol synthesis gas to methanol. Additionally in this embodiment, the chemical reaction arrangement of the apparatus may be operable to provide the stoichiometric condition (Eq. 4). Further, at the first stage for steam reforming the stoichiometric conditions may include but not limited to a pressure in a range of 10 Bar to 30 Bar, and a temperature in a range of 750° C. to 950° C. Furthermore, at the second stage of methanol synthesis the stoichiometric conditions may include but not limited to a pressure in a range of 50 Bar to 150 Bar, and a temperature in a range of 200° C. to 250° C. In practice, use of high temperature in the first stage for steam reforming the stoichiometric conditions is advantageous in terms of higher rate of reaction and removal of impurities present in feed received from the anaerobic digestion arrangement

In another embodiment, the apparatus for producing methanol from organic material may further include a methanol reformer for converting traces of Methane into Methanol received from purge stream of the chemical reaction arrangement. In this embodiment, the methanol reformer may include less exotic alloys/less active alloys as catalysts for converting traces of Methane into Methanol received from purge stream of chemical reaction arrangement. In practice, use of less exotic alloys/less active alloys as catalysts is advantageous in terms of reducing loss of methane due to recycling of the purge gasses.

In yet another embodiment, the chemical reaction arrangement of the apparatus may be operable to provide the stoichiometric condition (Eq. 4). In example, at the first stage for steam reforming the stoichiometric conditions may include but not limited to a pressure in a range of 10 Bar to 30 Bar, and a temperature in a range of 750° C. to 950° C. Further, at the second stage of methanol synthesis the stoichiometric conditions may include but not limited to a pressure in a range of 50 Bar to 150 Bar, and a temperature in a range of 200° C. to 250° C. In practice, use of less exotic alloys/less active alloys as catalyst at the second stage is advantageous in terms of reducing loss of methane due to recycling of the purge gasses and high yield of methanol.

In still another embodiment, the catalysts may include but not limited to nickel-alumina, nickel foil, copper and/or platinum.

In other exemplary embodiment, the method of using an apparatus for producing methanol from organic material may include receiving the organic material at an anaerobic digestion arrangement and anaerobically-digesting the organic material in oxygen-depleted conditions to generate methane gas, and reacting the methane gas with water vapour and carbon dioxide in a stoichiometric condition (Eq. 4) between methane steam reforming and methane dry reforming to generate methanol in the chemical reaction arrangement.

### DETAILED DESCRIPTION OF DIAGRAMS

Referring to FIG. 1, there is shown an illustration of an apparatus for producing methanol pursuant to the present disclosure. The apparatus is indicated generally by 10, and includes an anaerobic digestion arrangement 20 and a chemical reforming arrangement 30, wherein a biogas feed pipe arrangement 40 is operable to provide a flow of methane gas, in operation from the anaerobic digestion arrangement 20 to the chemical reforming arrangement 30. The anaerobic digestion arrangement 20 includes one or more anaerobic digestion vessels that are operable to provide for microorganism-based digestion of organic waste and/or organic materials under oxygen-depleted reaction conditions; the one or more anaerobic digestion vessels are, for example fabricated from seam-welded formed steel sheet, or similar. Moreover, the chemical reforming arrangement 30 includes one or more chemical reaction vessels, for example fabricated from seam-welded formed steel sheet, or similar; the one or more chemical reaction vessels are operable to accommodate the aforementioned first and second stages. Moreover, the apparatus 10 further includes a control arrangement 50 for controlling admission of gas components to an internal region of at least one reaction vessel of the chemical reforming arrangement 30, for example admission in operation of steam carbon dioxide and biogas into the at least one reaction vessel. Furthermore, a gas sensing arrangement 60, as described in the foregoing, is coupled to the at least one reaction vessel of the chemical reforming arrangement 30; the gas sensing arrangement 60 provides sensed gas concentration measurements (for example, p.p.m. concentration of carbon dioxide (CO₂) present in the at least one reaction vessel, p.p.m. concentration of methane (CH₄) present in the at least one reaction vessel, p.p.m. concentration of methanol (CH₃OH) present in the at least one reaction vessel, p.p.m. concentration of carbon monoxide (CO) present in the at least one reaction vessel, p.p.m. concentration of hydrogen (H₂) present in the at least one reaction vessel, p.p.m. concentration of water vapour (H₂O) present in the at least one reaction vessel) to the control arrangement 50 that employs an algorithm to control the admission of gas components to an internal region of at least one reaction vessel of the chemical reforming arrangement 30, for example to achieve a substantially stoichiometric reaction as aforementioned.

Referring next to FIG. 2, there is shown a method of operating the apparatus 10 of FIG. 1. In a first step S1 100 of the method, the method includes supplying organic material, for example agricultural waste, to the anaerobic digestion arrangement 20. In a second step S2 110 of the method, the method includes anaerobically digesting the supplied organic material to generate biogas, primarily methane. In a third step S3 120, the method includes using the control arrangement 50 to receive signals from the gas sensing arrangement 60 indicative of gas component concentrations present in the one or more chemical reaction vessels of the chemical reforming arrangement 30, to apply values corresponding to the received signals to a stochiometry control algorithm executed upon processing hardware of the control arrangement 50, to generate control signals from the stochiometry control algorithm and to apply the control signals to the biogas feedpipe arrangement 40 and to other sources of gases (for example, a carbon dioxide generator, a steam generator) to maintain an operating stochiometry within the one or more chemical reaction vessels (to maintain in operation a reaction condition as described by Equation 4 (Eq. 4). In a fourth step S4 130, the method includes extracting (for example, via a process of selective condensation) methanol from the one or pre chemical reaction vessels. The steps S1 to S4 are beneficially performed concurrently so that the apparatus 10 is capable of continuously generating methanol from organic waste and similar organic materials.

Modifications to embodiments of the invention described in the foregoing are possible without departing from the scope of the invention as defined by the accompanying claims. Expressions such as “including”, “comprising”, “incorporating”, “consisting of”, “have”, “is” used to describe and claim the present invention are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

1. An apparatus for producing methanol in a continuous manner from organic material, characterized in that the apparatus includes:
(i) an anaerobic digestion arrangement for receiving the organic material and for anaerobically-digesting the organic material in oxygen-depleted conditions to generate an anaerobic digestion gas (AD gas) comprising at least methane, and carbon dioxide;
(ii) a pressure swing absorption (PSA) arrangement for the removal of excess carbon dioxide;
(iii) a chemical reaction arrangement (30) for simultaneously reacting the methane gas with water vapour and carbon dioxide in a stoichiometric condition of reaction
CO₂+3CH₄+2H₂O=4CH₃OH
between methane steam reforming and methane dry reforming within at least one vessel to generate a synthesis gas, and converting the synthesis gas to methanol; and
(iv) a recovery arrangement for recovering unreacted methane and feeding the recovered unreacted methane into the exit stream from the anaerobic digestion arrangement.

2. The apparatus as claimed in claim 1, wherein the apparatus includes an arrangement for feeding hydrogen into the exit stream from the anaerobic digestion arrangement.

3. The apparatus as claimed in claim 2, wherein the arrangement for feeding hydrogen generates hydrogen by a photocatalytic process.

4. The apparatus as claimed in claim 1, wherein the stoichiometric condition is maintained using a control arrangement provided in operation with temperature sensing signals and gas component sensing signals indicative of operating conditions within the chemical reaction arrangement, for controlling rates of supply of the methane gas, water vapour and carbon dioxide into the chemical reaction arrangement.

5. The apparatus as claimed in claim 1, wherein the apparatus includes a renewable energy source for providing operating power to the chemical reaction arrangement.

6. The apparatus of as claimed in claim 1, wherein the chemical reaction arrangement is operable to employ a catalyst arrangement including, nickel, nickel-alumina, nickel foil, copper and/or platinum catalysts.

7. The apparatus as claimed in claim 1, wherein the chemical reaction arrangement is operable to provide the stoichiometric condition of reaction
CO₂+3CH₄+2H₂O=4CH₃OH:
(i) at a first stage for steam reforming at a pressure in a range of 10 Bar to 30 Bar, and at a temperature in a range of 750° C. to 950° C.; and
(ii) at a second stage of methanol synthesis at a pressure in a range of 50 Bar to 150 Bar, and at a temperature in a range of 200° C. to 250° C.

8. (canceled)

9. A method of using an apparatus for producing methanol in a continuous manner from organic material, wherein the method includes:
(i) receiving the organic material in an anaerobic digestion arrangement, and anaerobically-digesting the organic material in oxygen-depleted conditions to generate a gas comprising methane and carbon dioxide;
(ii) removing excess carbon dioxide in a pressure swing absorption (PSA) arrangement;
(iii) simultaneously the gas with water vapour and carbon dioxide in a stoichiometric condition of reaction
CO₂+3CH₄+2H₂O=4CH₃OH
between methane steam reforming and methane dry reforming within at least one vessel to generate a synthesis gas and converting the synthesis gas to methanol in a chemical reaction arrangement; and
(iv) recovering unreacted methane in a recovery arrangement and feeding the recovered unreacted methane into the exit stream from the anaerobic digestion arrangement.

10. The method as claimed in claim 9, wherein the method includes maintaining the stoichiometric condition using a control arrangement, provided in operation with temperature sensing signals and gas component sensing signals indicative of operating conditions within the chemical reaction arrangement, for controlling rates of supply of the methane gas, water vapour and carbon dioxide into the chemical reaction arrangement.

11. The method as claimed in claim 9, wherein the method includes using a renewable energy source for providing operating power to the chemical reaction arrangement.

12. The method as claimed in claim 9, wherein the method includes operating the chemical reaction arrangement to employ a catalyst arrangement including nickel-alumina, nickel foil, copper and/or platinum catalysts.

13. The method as claimed in claim 9, wherein the method includes operating the chemical reaction arrangement to provide the stoichiometric condition of reaction
CO₂+3CH₄+2H₂O=4CH₃OH:
(i) at a first stage for steam reforming at a pressure in a range of 10 Bar to 30 Bar, and at a temperature in a range of 750° C. to 950° C.; and
(ii) at a second stage of methanol synthesis at a pressure in a range of 50 Bar to 150 Bar, and at a temperature in a range of 200° C. to 250° C.

14. (canceled)

15. A computer program product comprising a non-transitory computer-readable storage medium having computer-readable instructions stored thereon, the computer-readable instructions being executable by a computerized device comprising processing hardware for executing a method as claimed in claim 9.
